(19) United States
(12) Patent Application Publication
(10) Pub. No.: US 2004/0081705 A1
(43) Pub. Date: Apr. 29, 2004

(54) Digestion promoter for ruminant animal and breeding method of ruminant animal

(76) Inventor: Mana Gotou, Aichi-ken (JP)

Correspondence Address: RADER, FISHMAN & GRAUER PLLC, David T. Nikaido, Suite 501, 1233 20th Street, N.W., Washington, DC, 20036, US

(21) Appl. No.: 10/663,727

(22) Filed: Sep. 17, 2003

(30) Foreign Application Priority Data
Sep. 18, 2002 (JP) 2002-270847

(51) Int. Cl. A61K 33/00

(52) U.S. Cl. 424/600

(57) ABSTRACT

A digestion promoter for a ruminant animal for promoting digestion of feed in a stomach of the ruminant animal, containing, as a main component, electrolyzed weak alkaline water produced in an electrolytic cell with an ion-permeable membrane. A breeding method of a ruminant animal, providing plants as feed for the ruminant animal with drinking water containing, as a main component, electrolyzed weak alkaline water produced in an electrolytic cell with an ion-permeable membrane.

### BACKGROUND OF THE INVENTION

1. Field of the Invention

The present invention relates to a digestion promoter for ruminant animals and a breeding method of ruminant animals.

2. Description of the Prior Art

For breeder, it is very important to breed domestic animals as quickly as possible in good physical condition thereby to enhance productivity of the domestic animals. For this purpose, investigation of feed indispensable for breeding of domestic animals has been made as proposed in Japanese Patent Laid-open Publication No. 6-153809. However, investigation of drinking water indispensable for breeding of domestic animals has not yet been undertaken.

### SUMMARY OF THE INVENTION

It is, therefore, a primary object of the present invention to provide a drinking water and a breeding method capable of breeding domestic animals as quickly as possible in good physical condition.

According to the present invention, the object is accomplished by providing a digestion promoter for ruminant animals such as cattle, sheep, goats and deer for promoting digestion of feed in the stomach of the ruminant animal, containing, as a main component, electrolyzed weak alkaline water produced in an electrolytic cell with an ion-permeable membrane.

According to an aspect of the present invention, there is provided a breeding method of ruminant animals, providing plants as feed for the ruminant animal with drinking water containing, as a main component, electrolyzed weak alkaline water produced in an electrolytic cell with an ion-permeable membrane.

As a result of a digestion experiment of dried goods in in-vitro using internal solution of the stomach of the ruminant animal (rumen solution), it has been found that a digestion rate of the dried goods in use of electrolyzed weak alkaline water (pH 8.5-10) for preparation of artificial saliva is increased more than that in use of distilled water for preparation of artificial saliva.

In a result of a gas production experiment of the dried goods using the rumen solution of the ruminant animal in in-vitro, it has been found that the gas production amount and velocity in use of the electrolyzed weak alkaline water for drinking water are increased more than those in use of usual water such as well water for drinking water. Such increase of the gas production amount and velocity means an increase of the digestion rate of the feed. From these facts, it has been confirmed that the electrolyzed weak alkaline water is a main component effective for promoting digestion of the feed of ruminant animals.

### BRIEF DESCRIPTION OF THE DRAWINGS

Other objects, features and advantages of the present invention will be more readily appreciated from the following detailed description of practical embodiments of the present invention when taken together with the accompanying drawings, in which:

FIG. 1 is a graph showing a gas production amount of timothy in relation to a period of time for culture in a gas production experiment;

FIG. 2 is a graph showing a gas production amount of alfalfa hay cube in relation to a period of time for culture in a gas production experiment;

FIG. 3 is a graph showing a gas production amount of napier grass in relation to a period of time for culture in a gas production experiment;

FIG. 4 is a graph showing a gas production amount of calopogonium mucunoides in relation to a period of time for culture in a gas production experiment; and

FIG. 5 is a graph showing a gas production amount of soy bean cake in relation to a period of time for culture in a gas production experiment.

### PREFERRED EMBODIMENT OF THE INVENTION

As a result of various investigations in respect of digestion of botanical feed for domestic animals, it has been found that electrolyzed weak alkaline water is useful for promoting digestion of feed in the stomach of a ruminant animal such as cattle, sheep, coats and deer. The electrolyzed alkaline water is produced by electrolysis of raw water such as tap water in the cathode chamber of an electrolytic cell with an ion-permeable membrane. In the electrolytic cell, weak alkaline water of pH 8.5-10.0 is, in general, produced by electrolysis of the raw water.

Used as feed for ruminant animals are rice straw, napier grass, calopogonium mucunoides, timothy, alfalfa bay cube and soy bean cake. In the present invention, a sheep was selected as the ruminant animal for digestion experiment of dried goods and gas production experiment of the dried goods in in-vitro using internal solution of rumen of the sheep. In the digestion experiment of dried goods, it has been confirmed that a digestion rate of the dried goods is significantly increased in use of electrolyzed weak alkaline water for preparation of artificial saliva in comparison with that in use of distilled water for preparation of the artificial saliva.

In the gas production experiment in in-vitro using internal solution of the rumen of sheep, it has been confirmed that an amount and a velocity of gas production in use of electrolyzed weak alkaline water for drinking water of sheep are increased more than those in use of well water for drinking water of the sheep. Such increase of the amount of gas production and the velocity of gas production in the experiment means an increase of the digestion rate of feed. Since the digestion experiment and gas production experiment of dried goods were carried out in the atmosphere similar to the rumen of the sheep, the result of the experiments represents a promotion tendency of feed in the rumen of cattle and sheep.

From the foregoing fact, it has been confirmed that the electrolyzed weak alkaline water is a main component of drinking water useful for promoting digestion of the feed in the rumen of ruminant animals. In this respect, it is considered that the electrolyzed weak alkaline water is effective to increase a dissolution degree of feed protein and a decomposition degree of hemicellulose and to cause change of fungus phase.

In a practical embodiment of the present invention, a digestion experiment of dried goods and a gas production experiment of the dried goods were adapted to a sheep selected as the ruminant, rice straw, napier grass, calopogonium, mucunoides, timothy, alfalfa hay cube and soy bean cake used as feed of the sheep, and electrolyzed weak alkaline water, distilled water and well water used as a sample water. The characteristics of the electrolyzed weak alkaline water and well water are shown in the following table 1.

TABLE 1
Solution for preparation:

| Characteristic | | |
|---|---|---|
| Component (mg/l) | Electrolyzed alkaline water | Well water |
| pH | 9.0 | 8.1 |
| DO | 6.0 | 6.5 |
| Na⁺ | 16.6 | 102.2 |
| Ca⁺ | 3.6 | 12.3 |
| K⁺ | 1.1 | 2.1 |
| Mg²⁺ | 1.9 | 8.8 |

(The character DO represents dissolved oxygen)

Experiment 1:

For a digestion experiment of dried goods in in-vitro, a sample feed of about 0.45 g was prepared by rice straw, napier grass and calopogonium mucunoides, and two kinds of McDougall artificial saliva shown in the following table 2 were prepared by internal solution of the rumen collected from the sheep and electrolyzed weak alkaline water and distilled water. The internal solution of rumen of 10 ml was added to the artificial saliva of 40 ml respectively to artificially prepare a rumen (culture medium), and the feed was cultured at 39° C. in the artificially prepared rumen for 48 hours.

After finish of the culture, the culture medium was filtrated by suction and separated to filtrated liquid and residue. The residue was dried by wind at 135° C. for two hours to obtain a dried good. Thus, a digestion rate of the dried good, [(A−B)/B]×100] %, was calculated on a basis of the weight (A) of the dried good and the weight (B) of the sample feed before culture thereof. The component of the artificial saliva is shown in the following table 2, the pH of the artificial saliva and respective culture medium are shown in the following table 3, and the digestion rate (%) of the dried good is shown in the following table 4.

TABLE 2
Component of Mc Dougall artificial saliva

| Component | Amount of component (g/l) |
|---|---|
| NaHCO₃ | 9.8 |
| KCL | 0.57 |
| CaCO₃ | 0.04 |
| NaHPO₄.12H₂O | 9.0 |
| NaCl | 0.47 |
| MgSO₄.7H₂O | 0.12 |

(Provided that, electrolyzed weak alkaline water and distilled water were used as the solution for preparation.)

TABLE 3
pH of artificial saliva and culture medium:

| | Solution for preparation | |
|---|---|---|
| Kinds of solution | Electrolyzed water | Distilled water |
| Artificial saliva | 8.31 | 8.28 |
| Culture medium of blank 1 | 7.45 | 7.80 |
| Culture medium of blank 2 | 8.65 | 7.75 |
| Culture medium of napier grass | 6.92 | 7.0 |
| Culture medium of rice straw | 6.92 | 6.95 |
| Culture medium of Calopo | 7.20 | 7.0 |

(Provided that, the electrolyzed water represents electrolyzed weak alkaline water.)

TABLE 4
Digestion rate of dried goods (%):

Solution for preparation

| Sample feed | Electrolyzed water | Distilled water |
|---|---|---|
| Napier | 70.1 | 62.9 |
| Rice straw | 56.0 | 45.3 |
| Calopo | 60.4 | 56.8 |

In the column of the kinds of solution of the table 3, the culture medium of blank represents an artificial rumen prepared by the artificial saliva and internal solution of the rumen, the culture medium of napier grass represents a culture medium of napier grass cultured in the artificial rumen for 48 hours immediately after the preparation thereof, the culture medium of rice straw represents a culture medium of rice straw cultured in the artificial rumen for 48 hours immediately after the preparation thereof, and the culture medium of Calopo represents a culture medium of calopogonium mucunoides cultured in the artificial rumen for 48 hours immediately after the preparation thereof.

As shown in the table 3, there is not any difference in pH between the artificial saliva prepared by the electrolyzed weak alkaline water and the artificial saliva prepared by the distilled water. In the artificial rumen (blank culture medium) prepared by the artificial saliva, the pH of the blank culture medium after lapse of 48 hours becomes high in non-hygienic extent. Transition of the property of rumen from its normal value to an alkaline value is called alkalosis affecting harmful influence to animals. Each pH of the three kinds of culture medium is, however, in a normal value of approximate pH 7. This is presumed by the fact that microorganism in the culture medium decomposes carbohydrate as in microorganism of rumen and acts as fermentation component to produce volatile fatty acid, lactic acid, methane and carbon dioxide.

In the culture medium prepared by the artificial saliva using the electrolyzed weak alkaline water and distilled water, the same fermentation as in the rumen was caused. Since the calopogonium mucunoides is a leguminous pasture (grass) containing a large amount of protein, the culture medium of calopogonium mucunoides is alkalized more than the other culture medium. This is presumed by the fact that microorganism in the culture medium contains decomposition enzyme of protein as in the rumen and decomposes the protein in the sample feed into ammonia, peptide and amino acid.

In the table 4 showing a result of the dry digestion rate, Napier represents napier grass, and Capolo represents calopogonium mucunoides. As shown in the table 4, it has been found that the digestion rate of the sample feed in the culture medium prepared by the artificial saliva using the electrolyzed weak alkaline water is significantly larger than that of the sample feed in the culture medium prepared by the artificial saliva using the distilled water.

Experiment 2:

For a gas production experiment in in-vitro, a sheep was selected as the ruminant animal on a basis of Menke & Steingass method, and timothy, alfalfa hay cube, napier grass, calopogonium mucuuoides and soy bean cake were used as feed of the sheep. In this experiment, electrolyzed weak alkaline water of pH 9.0 and well water were used for drinking water of the sheep, and internal solution of the rumen was collected from the sheep and mixed with a medium at a ratio of 2:1 (60 ml:30 ml) to prepare a culture medium. As shown in the following table 5, the medium was prepared by a mixture of reagents a˜e. In the preparation of the medium, the reagent a of 0.1 ml, the reagent b of 200 ml, the reagent c of 200 ml, the reagent d of 1.0 ml and the reagent e of 40 ml were mixed with water of 400 ml immediately before collection of the internal solution of the rumen and reduced with carbon dioxide (gas).

TABLE 5
Component of reagent:

| Reagent | Component | Amount | |
|---|---|---|---|
| Reagent a: | | | |
| Micromineral solution | CaCl₂.2H₂O | 13.2 | g |
| (/100 ml water) | MnCl₂.4H₂O | 10.0 | g |
| | CoCl₂.6H₂O | 1.0 | g |
| | FeCl₂.6H₂O | 3.0 | g |
| Reagent b: | | | |
| Rumen buffer solution | NH₄HCO₃ | 4.0 | g |
| (/100 ml water) | NaHCO₃ | 35.0 | g |
| Reagent c: | | | |
| Macromineral solution | NaHPO₄ | 5.7 | g |
| (/100 ml water) | KH₂PO₄ | 6.2 | g |
| | MgSo₄.7H₂O | 0.6 | g |
| Reagent d: | | | |
| Resazurin solution | | 0.1% | (w/v) |
| Reagent e: | | | |
| Reduction solution | IN NaOH | 4.0 | ml |
| | Na₂S.9H₂O | 625 | mg |
| | Water | 95 | ml |

For the gas production experiment, each sample feed of 0.45 g was cultured in the culture medium at 39° C. for 72 hours. During the culture, an amount of gas generation was measured at each lapse of 3 hours, 6 hours, 9 hours, 12 hours, 24 hours, 48 hours and 72 hours to calculate gas production parameters. In this experiment, microorganism in the rumen was alive in the culture medium for about 96 hours during which each sample feed was maintained in a fermetable condition. The gas production parameter was calculated by a formula G=B(1−e^{-c(t-L)} shown in Neway programme (Chen, 1997). In the formula, the character G represents an amount of gas generation, the character B represents an amount of latent gas production, the character c represents a gas production velocity coefficient (%/h), the character t represents a period of time (h) for culture, and the character L represents a retard time of fermentation.

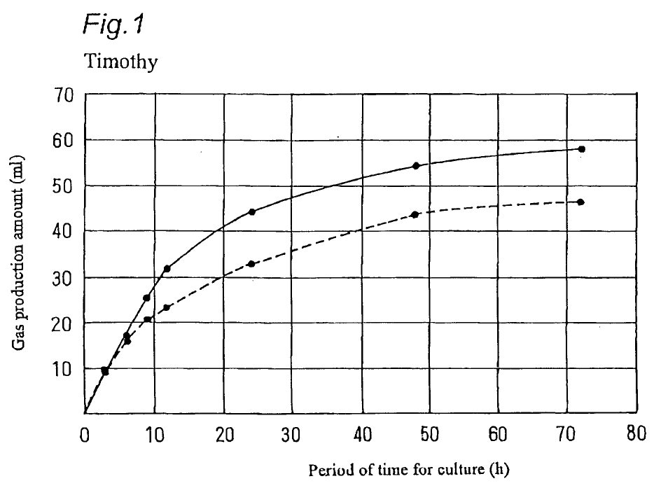

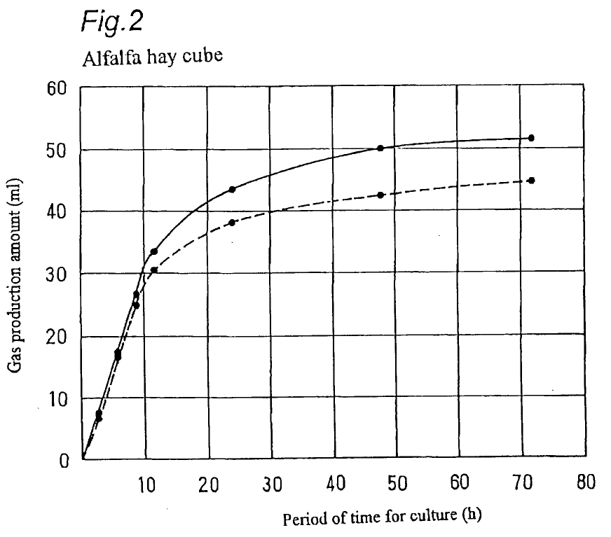

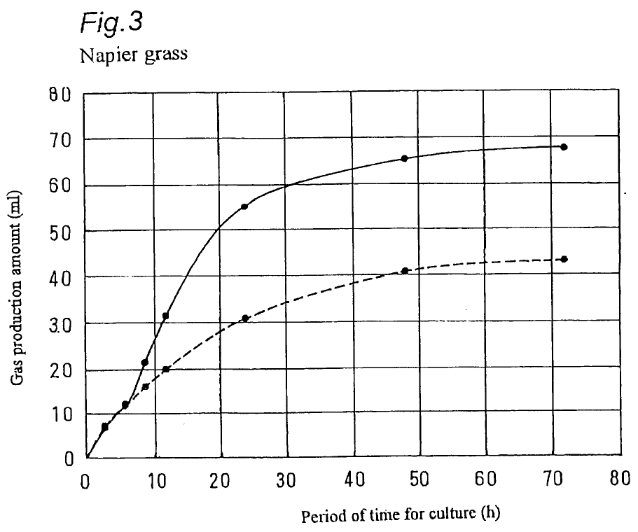

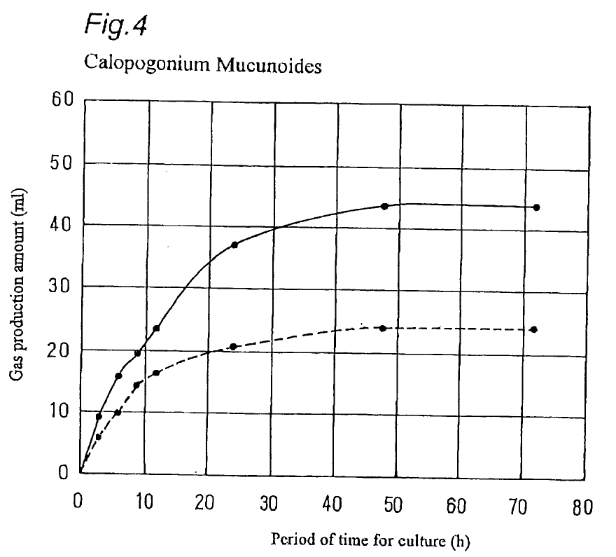

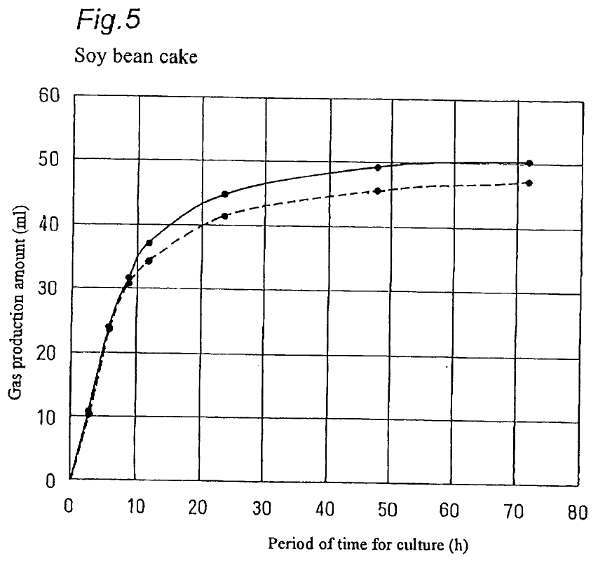

The gas production amount of each sample feed in relation to the period of time for culture is shown in FIGS. 1 to 5, and the gas production parameter is shown in FIG. 6. In FIGS. 1 to 6, each solid line represents a result of use of the electrolyzed weak alkaline water for drinking water, and each broken tine represents a result of use of the well water for drinking water.

TABLE 6
Gas production parameter:

| | Sample feed | B (%) | C (%/h) | L (t) |
|---|---|---|---|---|
| Drinking water | Timothy | 58.9 | 6.3 | 0.0 |
| Electrolyzed | Alfalfa | 50.9 | 9.5 | 1.2 |
| alkaline water | Napier | 58.7 | 5.8 | 1.3 |
| | Calopo | 46.0 | 7.1 | 0.4 |
| | Soy bean cake | 49.1 | 12.7 | 0.9 |
| Well water | Timothy | 48.3 | 4.6 | 0.1 |
| | Alfalfa | 43.7 | 12.0 | 1.4 |
| | Napier | 45.0 | 4.7 | 0.0 |
| | Calopo | 23.9 | 9.1 | 0.1 |
| | Soy bean cake | 45.3 | 13.4 | 0.8 |

Each result of the gas production experiment of timothy, alfalfa hay cube, napier grass, calopogonium mucunoides and soy bean cake is shown in FIGS. 1, 2, 3, 4 and 5.

As shown in the figures, it has been found that the gas production amount in use of the electrolyzed weak alkaline water for drinking water is increased more than that in use of the well water for drinking water. This means that the electrolyzed weak alkaline water is useful to promote digestion of organic substance such as the sample feeds. In the sample feeds, it has been found that the gas production amount of the rice plant is increased more than that of the leguminous plant.

As shown in the table 6, it has been found that the latent gas production amount (the gas parameter B) in use of the electrolyzed weak alkaline water for drinking water is increased more than that in use of the well water for drinking water. The latent gas production amount corresponds with a digestion rate of organic substance. Accordingly, it has been confirmed that the electrolyzed weak alkaline water is useful to promote digestion of the sample feeds in the rumen. In the sample feeds, it has been found that the latent gas production amount of the rice plant is increased more than that of the leguminous plant.

What is claimed is:

1. A digestion promoter for a ruminant animal for promoting digestion of feed in a stomach of the ruminant animal, containing, as a main component, electrolyzed weak alkaline water produced in an electrolytic cell with an ion-permeable membrane.

2. A breeding method of a ruminant animal, providing plants as feed for the ruminant animal with drinking water containing, as a main component, electrolyzed weak alkaline water produced in an electrolytic cell with an ion-permeable membrane.
